(19) United States
(12) Patent Application Publication
(10) Pub. No.: US 2024/0237930 A1
(43) Pub. Date: Jul. 18, 2024

(54) SUBJECTING TEXTUAL MESSAGES SENT BY PATIENTS TO A MACHINE LEARNING MODEL TRAINED TO PREDICT WHETHER THEY ARE SUFFERING OR WILL SOON SUFFER FROM PSYCHOSIS

(71) Applicant: Providence St. Joseph Health, Seattle, WA (US)

(72) Inventors: Syneva J. Runyan, Kotzebue, AK (US); Billy Jackson, Olympia, WA (US)

(21) Appl. No.: 18/156,249

(22) Filed: Jan. 18, 2023

(51) Int. Cl. A61B 5/16; G06N 20/00; G16H 50/20

(52) U.S. Cl. CPC A61B 5/16; G06N 20/00; G16H 50/20; G06F 40/30

(57) ABSTRACT

A facility for predicting the presence of a mental health condition is described. The facility accesses a textual message originated by a sender, and applies to the textual message a machine learning model trained to predict whether the sender suffers from a distinguished mental health condition. In response to predicting that the sender suffers from the distinguished mental health condition, the facility takes an action with respect to the sender.

### BACKGROUND

Psychosis is a condition that affects the way a person's brain processes information. It causes the person to lose touch with reality. The person might see, hear, or believe things that aren't real.

### BRIEF DESCRIPTION OF THE DRAWINGS

FIG. 1 is a block diagram showing some of the components typically incorporated in at least some of the computer systems and other devices on which the facility operates.

FIG. 2 is a data flow diagram showing operation of the facility to train a psychosis detection model for detecting present and/or future psychosis.

FIG. 3 is a flow diagram showing the process performed by the facility in some embodiments to train each psychosis detection model.

FIG. 4 is a data flow diagram showing the facility's prediction of psychosis based upon a textual message.

FIG. 5 is a flow diagram showing a process performed by the facility in some embodiments to perform a psychosis prediction. In act 501, the facility intercepts a patient's textual message.

FIG. 6 is a display diagram showing a sample display presented by the facility to receive a message from a patient to a caregiver in some embodiments.

FIG. 7 is a display diagram showing a sample display presented by the facility in some embodiments to notify the patient of a positive psychosis prediction.

### DETAILED DESCRIPTION

When promptly detected and diagnosed and properly treated, individuals with mental illnesses such as psychosis do not pose any increased risk of violence over the general population. However, studies have found people experiencing psychosis are between 2-5 times as likely to have contact with the criminal justice system in comparison to control groups. Additionally, psychotic experiences are associated with significantly increased odds of subsequent suicidal ideation, suicide attempts, and suicide death, and there is a high proportion of young people with first-episode psychosis who attempted suicide before their first contact with mental health services.

The inventor has recognized that, if psychosis could be detected more promptly, patients could obtain the help they need more quickly, with less adverse impact on both themselves and others.

She noted that people increasingly engage in textual electronic communication with members of their medical care team, such as doctors, nurses, physician assistants, therapists, physical therapists, schedulers, etc. These communications can be via patient portals—accessed using the web or smartphone apps—via SMS messaging, via Internet email, or via a variety of other text messaging modalities.

In response to her recognition, the inventor conceived and reduced to practice a software and/or hardware facility for subjecting textual messages sent by patients to a machine learning model trained to predict whether they are suffering or will soon suffer from psychosis (“the facility”). In various embodiments, the facility takes a variety of actions in response to detecting that the patient is suffering from psychosis, including sending a message to a physician or other care team member for the patient; updating a dashboard presented to a physician or other care team members with this information; sending a message to the patient advising that they seek care; sending a similar message to a family member of the patient; updating the patient's electronic medical record (“EMR”) record, etc.

In some embodiments, in addition to the message contents, the facility submits to the model supplemental data relating to the message, such as metadata for the same message (e.g., day of week, time of day, provider role, subject line contents, indication of urgency), as well as other supplemental data (e.g., contents of the last 5 messages sent by the patient, contents of all of the messages sent by the patient in the past week, fields from the patient's EMR record such as test results, procedures performed, etc.).

In some embodiments, the facility sources training data for the model, such as by accessing the EMR records of many patients, and constructing a training observation for each in which a positive or negative psychosis diagnosis result for the patient is the dependent variable, and a message sent by the patient and its supplemental data. In various embodiments, the facility performs periodic or continuous retraining of the model to expand the set of observations on which the trained model state is based, update the model to reflect more recent observations, incorporate new patterns of diagnosis signal, etc.

In some embodiment, the facility maintains a set of models, each trained from and applied to a different population, such as different populations differentiated on the basis of one or more of age, geographic location, urban vs. rural locale, race or ethnic background, education level, sex, other medical or mental health conditions, etc. In some embodiments, as a way of identifying a new model that should be trained from and applied to a particular population, the facility begins by applying existing models of the set to each population, and evaluating the accuracy of the resulting predictions. For each population, if the accuracy of the predictions for that population by a particular one of the existing set of models is high enough to be adequate, then members of this population are assigned to that model for evaluation; on the other hand, if none of the existing set of models produces an adequately high level of accuracy, the facility establishes and trains a new model of the set for application to that population. In various embodiments, the facility pursues various other approaches to reducing the bias of existing models of the set with respect to particular populations for which the model's accuracy was low.

By operating in some or all of the ways described above, the facility reduces the average amount of time it takes for patients to be diagnosed and receive treatment for psychosis, thus reducing negative impacts on the patients and others.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by automatically updating a patient's EMR record to contain a positive psychosis prediction alert, the facility precludes the use of processing resources that would have been needed to operate a user interface used by a care team member to manually add such an alert to the patient's EMR record. Similarly, by automatically sending a positive psychosis prediction alert to the patient and/or a family member, the facility precludes the use of processing resources that would have been needed to operate a user interface used by a care team member to manually send such alerts.

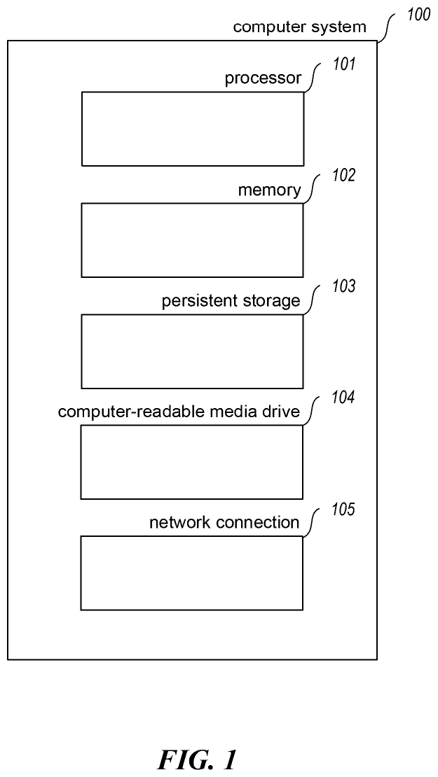

FIG. 1 is a block diagram showing some of the components typically incorporated in at least some of the computer systems and other devices on which the facility operates. In various embodiments, these computer systems and other devices 100 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 101 for executing computer programs and/or training or applying machine learning models, such as a CPU, GPU, TPU, NNP, FPGA, or ASIC; a computer memory 102 for storing programs and data while they are being used, including the facility and associated data, an operating system including a kernel, and device drivers; a persistent storage device 103, such as a hard drive or flash drive for persistently storing programs and data; a computer-readable media drive 104, such as a floppy, CD-ROM, or DVD drive, for reading programs and data stored on a computer-readable medium; and a network connection 105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

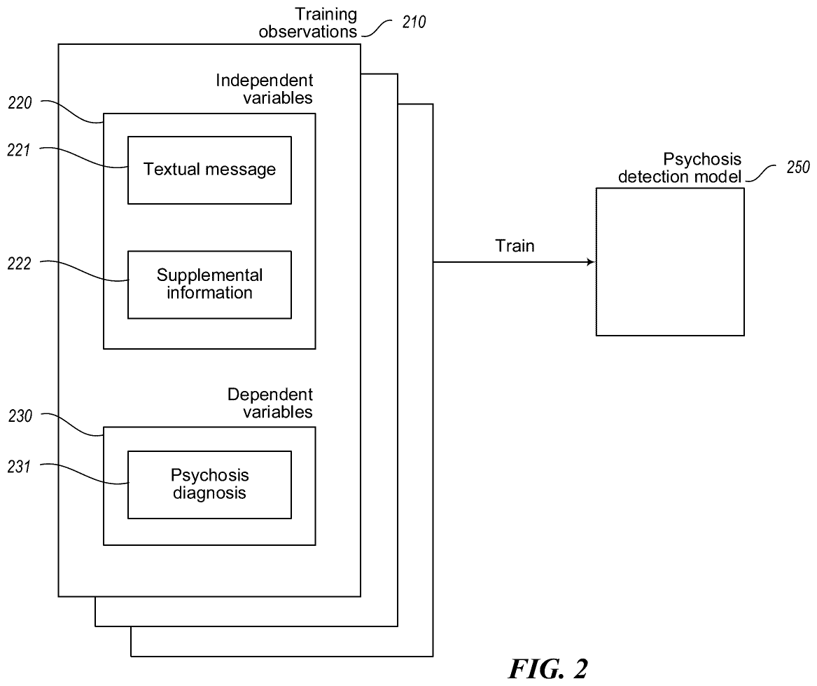

FIG. 2 is a data flow diagram showing operation of the facility to train a psychosis detection model for detecting present and/or future psychosis. In some embodiments, the psychosis detection model 250 is a compound model that first performs primary component analysis (“PCA”), then uses linear regression on the results of the PCA to predict psychosis. The design and training of machine learning models of this type is discussed in the following articles, each of which is herein incorporated by reference in its entirety: (1) Advait Thergaonkar, Merging Principal Component Analysis (PCA) with Artificial Neural Networks, Published in Analytics Vidhya, Sep. 22, 2020, available at medium.com/analytics-vidhya/merging-principal-component-analysis-pca-with-artificial-neural-networks-1ea6dad2c095; (2) Wikipedia, Principal component regression, available at en.wikipedia.org/wiki/Principal_component_regression; (3) Kenneth Leung, Principal Component Regression—Clearly Explained and Implemented, Towards Data Science, available at towardsdatascience.com/principal-component-regression-clearly-explained-and-implemented-608471530a2f; and (4) Ewa Sobolewska, Principal Component Regression, RPubs, available at rpubs.com/esobolewska/pcr-step-by-step. In cases where the present application and a document incorporated by reference disagree, the present application controls. In various embodiments, the facility uses a variety of other machine learning model types, such as various neural network architectures.

The facility uses training observations 210 to train the psychosis detection model. Each training observation corresponds to a different person about whom a positive or negative psychosis diagnosis and at least one contemporaneous message are available, and includes both independent variables 220, and dependent variables 230 whose value the model is being trained to infer from the independent variables. In some embodiments, the facility retrieves the contents of each training observation from the EMR record of the person to whom it corresponds. The independent variables include a textual message 221 sent by this person to members of the person's care team, as well as supplemental information 222 about the message and/or the person. In various embodiments, the version of the textual message incorporated in the independent variables include the full contents of the textual message; metrics and other attributes derived from the contents of the textual message, such as its semantic density, or the extent to which it uses “sound words;” or both. In various embodiments, the supplemental information can include a variety of combinations of elements, such as a day of the week on which the message was sent, a time of day in which the message was sent, a URL of the addressee to whom the message was sent, a subject of the message, an urgency level of the message, contents of one or more other messages contemporaneously sent by the sender, and/or other information about the person retrieved from their EMR record, such as procedures performed, test results, observations, provider notes, etc. In some embodiments, the dependent variables include a positive or negative psychosis diagnosis 231 made for the person, within a predetermined period of time of the sending of the textual message included among the independent variables.

In some embodiments, the facility segregates the training observations into different groups each representing a different population of people, for use in training separate models for those populations to use in performing a psychosis detection prediction for a person in a particular population. In some embodiments, the training is performed periodically in order to capture in the model's operation new informational trends present among the set of people used to generate the training observations, and/or extend the training base for the model to make its predictions more consistent and accurate.

In some embodiments, the facility's training of the model seeks to capture a variety of particular features, in some cases including one or more of measures of semantic density within the message or portions of it; references to voices or sounds that occur in the message; information about the timing of the message's sending; and/or patterns occurring among the message and preceding messages.

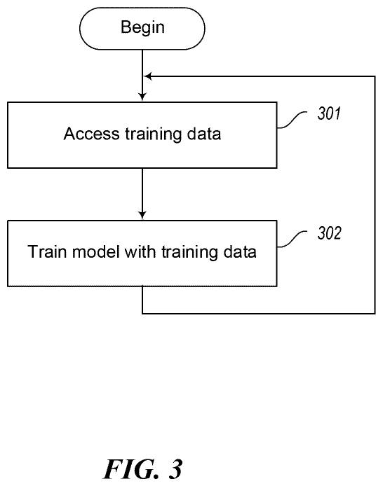

FIG. 3 is a flow diagram showing the process performed by the facility in some embodiments to train each psychosis detection model. In act 301, the facility accesses training data 210. In act 302, the facility trains the model 250 using the training data. After act 302, the facility continues in act 301 to perform a future retraining of the model.

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

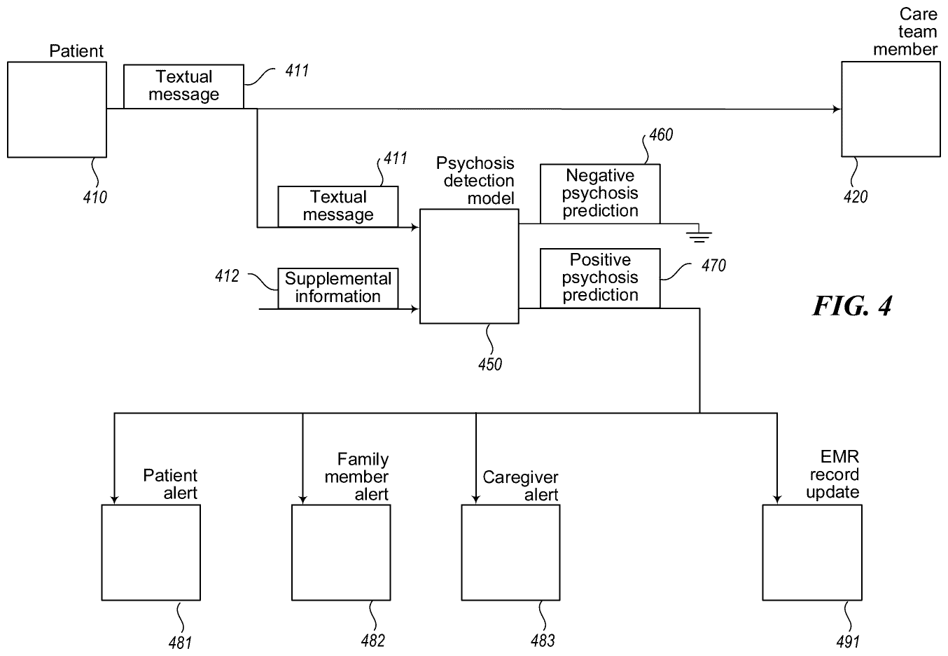

FIG. 4 is a data flow diagram showing the facility's prediction of psychosis based upon a textual message. The patient 410 creates and/or sends a textual message 411 to a care team member 420, such as the patient's primary care physician. An intercepted copy of the message, plus supplemental information 421, is subjected to the facility's psychosis detection model 450 to produce a psychosis prediction: either a negative prediction 460 reflecting that the patient's message and associated information do not demonstrate indicators of psychosis, or a positive psychosis prediction 470 indicating that it is likely that the patient is presently suffering from psychosis, or will suffer from psychosis in the near future. In some embodiments, the prediction made by the model is binary; in some embodiments, the prediction is a more continuous probability, against which the facility applies a probability threshold to discern negative predictions from positive ones. In response to a positive psychosis prediction, the facility causes alerts to be delivered to relevant people, such as a patient alert 481 that the facility causes to be presented at a patient; a family member alert 482 that the facility causes to be presented to a designated family member of the patient; and/or a caregiver alert 483 that the facility causes to be presented to one or more members of the patient's medical care team, or another person designated to follow up with patients likely to be suffering from psychosis. In some embodiments, the facility further performs an EMR record update 491 with respect to the patient, adding an indication to the patient's EMR record that a positive psychosis prediction has been produced for the patient.

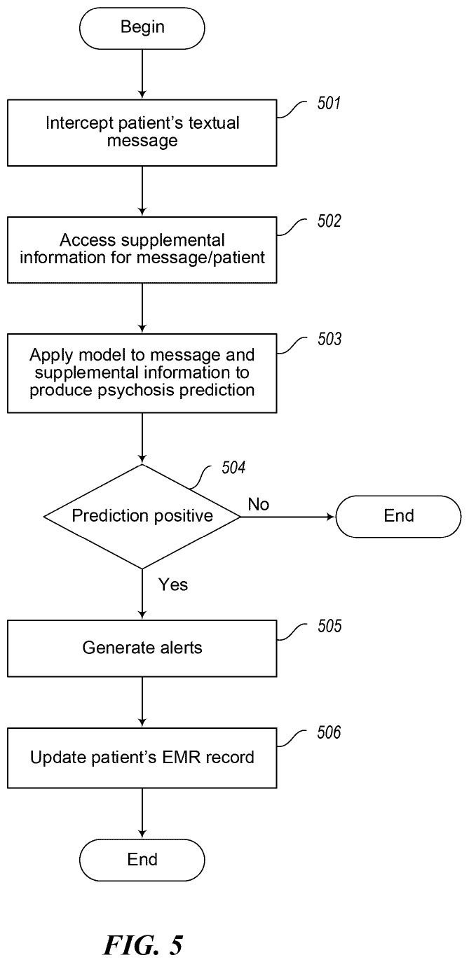

FIG. 5 is a flow diagram showing a process performed by the facility in some embodiments to perform a psychosis prediction. In act 501, the facility intercepts a patient's textual message. In various embodiments, the facility uses various techniques to intercept the textual message that are each suited to the modality of the message. As examples, in various embodiments, the facility performs the interception in the patient's message client; in the addressee's message client; at an intermediate point in the delivery of the message between these clients; or from a repository of already sent and/or already delivered messages. In act 502, the facility accesses supplemental information for the message and/or the patient, in some cases retrieving at least some of the supplemental information from the patient's EMR record. In act 503, the facility applies the trained model to the message and supplemental information to produce a psychosis prediction. In act 504, if the prediction is positive, then the facility continues in act 505, else this process concludes. In act 505, the facility generates alerts for presentation to one or more relevant people. In act 506, the facility updates the patient's EMR record with an indication that a positive psychosis prediction was produced for patient. After act 506, this process concludes.

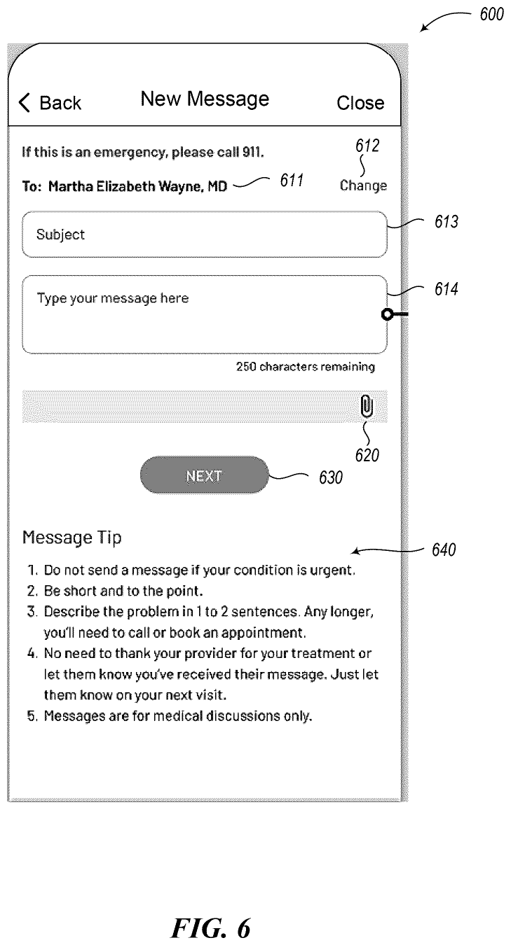

FIG. 6 is a display diagram showing a sample display presented by the facility to receive a message from a patient to a caregiver in some embodiments. The display 600 includes an addressee 611—such as the patient's primary care physician—and control 612 that the patient can activate in order to select a different or additional addressee. The display also includes a subject field 613 into which the user can enter a subject, such as by typing, swiping, pasting, or verbally dictating the text. The user may similarly enter the message into message field 14.

The user can activate file attachment control 620 in order to attach a file relevant to the message. In some embodiments, this attachment—such as an image—is used an input to the model, or information derived from it is, such as by identification of visual objects appearing in an image. The user can activate next control 630 in order to send the message, which has the effect of causing the psychosis prediction model to be applied to the message to perform a psychosis prediction.

While FIG. 6 and each of the display diagrams discussed below show a display whose formatting, organization, informational density, etc., is best suited to certain types of display devices, those skilled in the art will appreciate that actual displays presented by the facility may differ from those shown, in that they may be optimized for particular other display devices, or have shown visual elements omitted, visual elements not shown included, visual elements reorganized, reformatted, revisualized, or shown at different levels of magnification, etc.

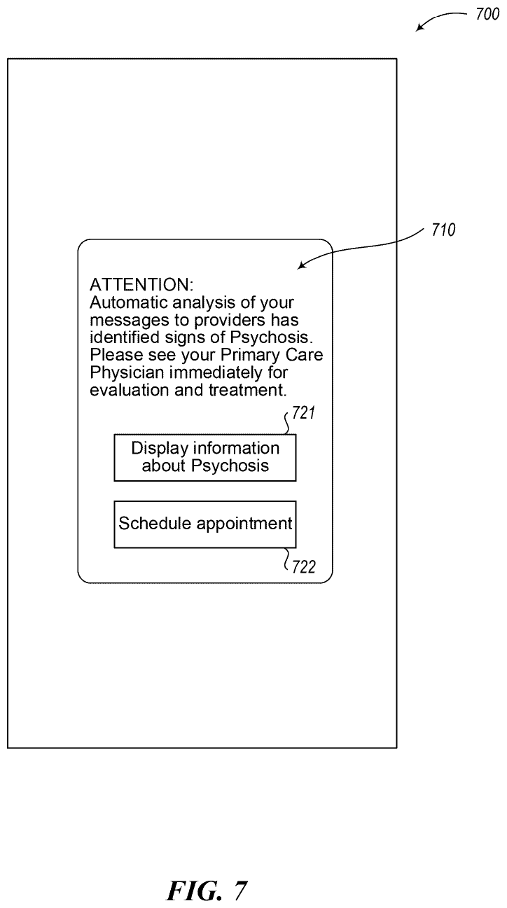

FIG. 7 is a display diagram showing a sample display presented by the facility in some embodiments to notify the patient of a positive psychosis prediction. The display 700 includes text 710 informing the patient of the positive prediction, and recommending that the patient immediately seek medical assistance. The display also includes a more information control 721 that the patient can activate in order to display additional information about psychosis. In some embodiments, the display includes a schedule appointment control that the patient can activate in order to schedule an appointment to discuss the psychosis prediction, such as in-person appointment, a video telehealth appointment, or a voice or text consultation.

The various embodiments described above can be combined to provide further embodiments. All of the U.S. patents , U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
intercepting a textual message originated by a sender;
accessing supplemental data relating to the textual message and/or the sender;
applying to the textual message and the supplemental data a machine learning model trained to predict whether the sender suffers from psychosis; and
in response to predicting that the sender suffers from psychosis, taking an action with respect to the sender.

2. The method of claim 1, further comprising:
initializing a machine learning model;
constructing first training data observations each corresponding to a different person in a first set of people and comprising (1) a dependent variable comprising a positive or negative psychosis diagnosis of the person, and (2) independent variables comprising (a) a textual message originated by the person and (b) supplemental data relating to the textual message and/or the person; and
at a first time, using the constructed first training observations to train the machine learning model to predict a person's psychosis diagnosis from textual message and supplemental data.

3. The method of claim 2, further comprising:
constructing second training data observations each corresponding to a different person in a second set of people distinct from the first set of people and comprising (1) a dependent variable comprising a positive or negative psychosis diagnosis of the person constituting a dependent variable, and (2) independent variables comprising (a) a textual message originated by the person and (b) supplemental data relating to the textual message and/or the person; and
at a second time that is later than the first time, using the constructed second training observations to further train the machine learning model to predict a person's diagnosis from textual message and supplemental data.

4. The method of claim 1 wherein the action comprises causing an alert to be presented to at least one person selected from among the sender, a family member of the sender, and/or a medical provider of the sender.

5. The method of claim 1 wherein the action comprises causing an EMR record corresponding to the sender to be updated to contain an indication that the sender is predicted to suffer from psychosis.

6. The method of claim 1 wherein the supplemental information comprises one or more of:
a day of the week on which the message was sent;
a time of day at which the message was sent;
a role of the addressee to whom the message was sent;
a subject of the message;
an urgency level of the message;
contents of one or more other messages recently sent by the sender; and/or information retrieved from an EMR record corresponding to the sender.

7. One or more instances of computer-readable media collectively having contents configured to cause a computing system to perform a method, the method comprising:
accessing a textual message originated by a sender;
applying to the textual message a machine learning model trained to predict whether the sender suffers from a distinguished mental health condition; and
in response to predicting that the sender suffers from the distinguished mental health condition, taking an action with respect to the sender.

8. The one or more instances of computer-readable media of claim 7 wherein the distinguished mental health condition is psychosis.

9. The one or more instances of computer-readable media of claim 7, the method further comprising:
initializing a machine learning model;
constructing first training data observations each corresponding to a different person in a first set of people and comprising (1) a dependent variable comprising a positive or negative diagnosis of the distinguished mental health condition for the person, and (2) independent variables comprising (a) a textual message originated by the person and (b) supplemental data relating to the textual message and/or the person; and
at a first time, using the constructed first training observations to train the machine learning model to predict a person's diagnosis of the distinguished mental health condition from textual message and supplemental data.

10. The one or more instances of computer-readable media of claim 9, the method further comprising:
for each person in the first set of people:
retrieving from an EMR record corresponding to the person the positive or diagnosis of the distinguished mental health condition that comprises the person's first training data observation.

11. The one or more instances of computer-readable media of claim 9, the method further comprising:
constructing second training data observations each corresponding to a different person in a second set of people distinct from the first set of people and comprising (1) a dependent variable comprising a positive or negative diagnosis of the distinguished mental health condition for the person, and (2) independent variables comprising (a) a textual message originated by the person and (b) supplemental data relating to the textual message and/or the person; and
at a second time that is later than the first time, using the constructed second training observations to further train the machine learning model to predict a person's diagnosis from textual message and supplemental data.

12. The one or more instances of computer-readable media of claim 7, the method further comprising:
accessing supplemental data relating to the textual message and/or the sender, and wherein the machine learning model is applied to the supplemental data in addition to the textual message to predict whether the sender suffers from the distinguished mental health condition.

13. The one or more instances of computer-readable media of claim 12 wherein the supplemental information comprises one or more of:
a day of the week on which the message was sent;
a time of day at which the message was sent;
a role of the addressee to whom the message was sent;
a subject of the message;
an urgency level of the message;
contents of one or more other messages recently sent by the sender; and/or information retrieved from an EMR record corresponding to the sender.

14. One or more instances of computer-readable media collectively containing a trained machine learning model data structure embodying a trained machine learning model, the data structure comprising:
a first trained state of the machine learning model that configures the machine learning model to predict, on the basis of a textual message originated by a person and supplemental information relating to the textual message and/or the person, whether the person is suffering from psychosis, such that the contents of the data structure are usable in applying the machine learning model to a distinguished textual message originated by a distinguished person and distinguished supplemental information relating to the distinguished textual message and/or the distinguished person to predict whether the distinguished person is suffering from psychosis.

15. The one or more instances of computer-readable media of claim 14 wherein the machine learning model comprises:
a neural network; or
a linear regression network configured to perform primary component analysis.

16. The one or more instances of computer-readable media of claim 14 the machine learning model one or more the following features:
semantic density within the message;
references to voices or sounds within the message;
timing of the message; and/or patterns occurring among the message and preceding messages.

17. The one or more instances of computer-readable media of claim 14 wherein the first trained state of the machine learning model has replaced a second trained state of the machine learning model on the basis of machine learning model retraining.

18. The one or more instances of computer-readable media of claim 14 wherein the first trained state of the machine learning model has been trained using information about people in a first differentiated population, the data structure further comprising a second trained state of the machine learning model that has been trained using information about people in a second differentiated population distinct from the first differentiated population.

19. The one or more instances of computer-readable media of claim 18 wherein the second differentiated population differs from the first differentiated population on the basis of one or more of:
age;
geographic location;
urban versus rural locale;
racial background;
ethnic background;
education level;
sex;
another medical condition; and/or another mental health condition.
